(19) United States
(12) Patent Application Publication
(10) Pub. No.: US 2007/0208072 A1
(43) Pub. Date: Sep. 6, 2007

(54) Maleate salt of tegaserod and crystalline forms thereof

(76) Inventors: Gustavo Frenkel, Beer Sheva (IL); Santiago Ini, Haifa (IL); Tamas Koltai, Netanya (IL)

Correspondence Address: KENYON & KENYON LLP, ONE BROADWAY, NEW YORK, NY, 10004, US

(21) Appl. No.: 11/655,510

(22) Filed: Jan. 18, 2007

Related U.S. Application Data
(60) Provisional application No. 60/760,306, filed on Jan. 18, 2006.
(60) Provisional application No. 60/872,950, filed on Dec. 4, 2006.

(51) Int. Cl. A61K 31/405; C07D 209/14

(52) U.S. Cl. 514/419; 548/503

(57) ABSTRACT

Provided is sesqui-tegaserod maleate and processes for preparation thereof.

### RELATED APPLICATIONS

This application claims the benefit of priority to U.S. Provisional Application Nos. 60/760,306, filed Jan. 18, 2006; and 60/872,950, filed Dec. 4, 2006, the contents of which are incorporated herein by reference.

### FIELD OF THE INVENTION

The present invention relates to solid state chemistry of a maleate salt of tegaserod.

### BACKGROUND OF THE INVENTION

Tegaserod maleate is an aminoguanidine indole 5HT4 agonist for the treatment of irritable bowel syndrome (IBS). Tegaserod maleate has the following structure:

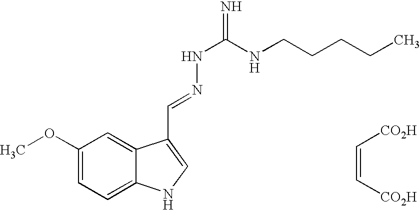

According to the prescribing information (Physician's Desk Reference, 57^{th} Ed., at Page 2339), tegaserod as the maleate salt is a white to off-white crystalline powder and is slightly soluble in ethanol and very slightly soluble in water. Tegaserod maleate is available commercially as ZELNORM®, in which it is present as crystalline form.

Tegaserod maleate is disclosed in U.S. Pat. No. 5,510,353 and in its equivalent EP 0 505 322 (example 13), and is reported to have a melting point of 190° C. (table 1 example 13).

The literature (Buchheit K. H, et al., J. Med. Chem., 1995, 38, 2331) describes a general method for the condensation of aminoguanidines with indole-3-carbadehydes in methanol in the presence of HCl (pH 3-4). The product obtained after solvent evaporation may be converted to its hydrochloride salt by treatment of the methanolic solution with diethylether/HCl followed by recrystallization from methanol/diethylether. Tegaserod base prepared according to this general method is characterized solely by a melting point of 155° C. (table 3 compound 5b). Additional Tegaserod maleate characterization was done by ¹H and ¹³C-NMR according to the literature (Jing J. et. al., Guangdong Weiliang Yuansu Kexue, 2002, 9/2, 51).

WO 04/085393 discloses four crystalline forms of tegaserod maleate. The search report for WO 04/085393 further identifies WO 00/10526, and Drugs Fut. 1999, 24(1) which provides an overview for tegaserod maleate. Additional crystalline forms of tegaserod maleate are provided in WO 2005/058819, one of which is characterized by an X-ray Diffraction pattern having peaks at 15.7, 16.9, 17.2, 24.1, 24.6 and 25.2±0.2 two theta (designated as Form B in that PCT publication).

The solid state physical properties of tegaserod salt may be influenced by controlling the conditions under which tegaserod salt is obtained in solid Form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state Form of a compound may also affect its behavior on compaction and its storage stability.

These practical physical characteristics are influenced by the conformation and orientation of molecules in the unit cell, which defines a particular polymorphic Form of a substance. The polymorphic form may give rise to thermal behavior different from that of the amorphous material or another polymorphic Form. Thermal behavior is measured in the laboratory by such techniques as capillary melting point, thermogravimetric analysis (TGA) and differential scanning calorimetry (DSC) and may be used to distinguish some polymorphic forms from others. A particular polymorphic Form may also give rise to distinct spectroscopic properties that may be detectable by powder X-ray crystallography, solid state C NMR spectrometry and infrared spectrometry.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

The polymorphic forms may further help in purification of tegaserod, particularly if they possess high crystallinity. In the event of metastability, a metastable polymorphic form may be used to prepare a more stable polymorph. Hence, discovery of new polymorphic forms and new processes help in advancing a formulation scientist in preparation of tegaserod as an active pharmaceutical ingredient in a formulation.

The present invention provides an additional polymorphic form of a maleate salt of tegaserod.

### SUMMARY OF THE INVENTION

In one embodiment, the present invention provides sesqui-tegaserod maleate, including in isolated and crystalline forms.

In another embodiment, the present invention provides sesqui-tegaserod maleate is in crystalline form which is characterized by a powder XRD pattern with peaks at about 3.9, 5.1, 5.4, and 14.0±0.2 degrees 2-theta.

In another embodiment, the present invention provides a process for preparing any of the above crystalline forms comprising providing tegaserod base in water; extracting the tegaserod base with ethyl acetate for more than one hour to obtain a mixture; combining the mixture with an aqueous solution of maleic acid.

In another embodiment, the present invention provides a process for preparing tegaserod maleate comprising converting sesqui-tegaserod maleate to tegaserod maleate.

In another embodiment, the present invention provides a process for preparing crystalline tegaserod maleate characterized by an X-ray Diffraction pattern having peaks at 15.7, 16.9, 17.2, 24.1, 24.6 and 25.2±0.2 two theta, comprising combining sesqui-tegaserod maleate with n-propanol and maleic acid, and maintaining the slurry to obtain the crystalline form.

### BRIEF DESCRIPTION OF THE FIGURES

FIG. 1 shows the powder XRD pattern of sesqui-tegaserod maleate Form H2

### DETAILED DESCRIPTION OF THE INVENTION

As used herein, the term “water content” refers to the content of water based upon the Karl Fisher assay for determining water content or thermogravimetric analysis (TGA). All percentages herein are by weight unless otherwise indicated.

The present invention provides sesqui-tegaserod maleate. The tegaserod content by HPLC assay of tegaserod (disclosed in WO 2005/058819) is 77.1%, and the maleate content by titration of maleic acid is 19.8%. Accordingly, the sesqui-tegaserod maleate comprises three molecules of tegaserod and two molecules of maleate. Compared with tegaserod base, the sesqui-tegaserod maleate is easier to isolate and purify.

The present invention provides sesqui-tegaserod maleate hydrate, preferably, the sesqui-tegaserod maleate is dihydrate, A Karl Fisher analysis of the sesqui-tegaserod maleate hydrate shows a water content of about 2.95%. Therefore, the sesqui-tegaserod maleate comprises three molecules of tegaserod, two molecules of maleate and two molecules of water.

The present invention further provides a solid crystalline form of sesqui-tegaserod maleate characterized by a powder XRD pattern with peaks at about 3.9, 5.1, 5.4 and 14.0±0.2 degrees 2-theta. In one embodiment, the characteristic peaks are about 3.9, 5.1, 5.4, 14.0 and 27.0±0.2 degrees 2-theta. This solid crystalline form of sesqui-tegaserod maleate hydrate is designated Form H2.

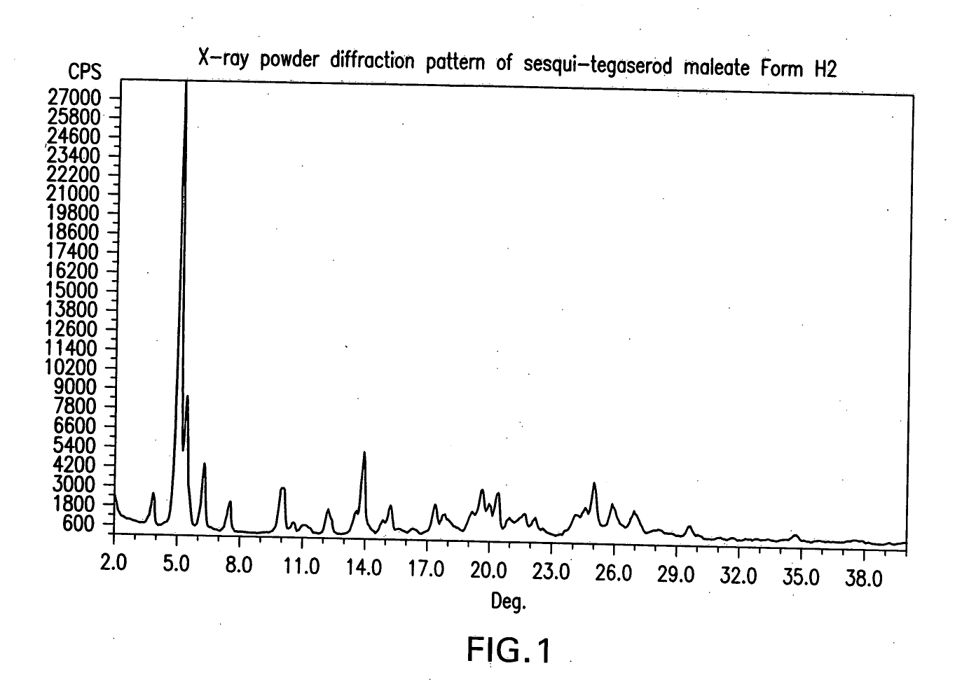

Form H2 may be further characterized by a powder XRD pattern with peaks at about 10.6, 13.6, 16.5, 22.8 and 29.7±0.2 degrees 2-theta (substantially as depicted in FIG. 1). Form H2 is preferably a hydrate.

Sesqui-tegaserod maleate Form H2 has a maximum particle size of about 500 μm. Preferably, Form H2 has a particle size of less than about 300 μm, more preferably less than about 200 μm, even more preferably less than about 100 μm, and most preferably less than 50 μm.

Particle size measurements may be performed by the following methods: sieves, sedimentation, electrozone sensing (coulter counter), microscopy, Low Angle Laser Light Scattering (LALLS).

Form H2 may be present in a batch or composition with a polymorphic purity of at least about 90% by weight.

The present invention also provides processes for preparing sesqui-tegaserod maleate Form H2 by providing tegaserod base in water; extracting the tegaserod base with ethyl acetate for more than one hour to obtain a mixture; combining the mixture with an aqueous solution of maleic acid to obtain Form H2. In one embodiment, the extraction with ethyl acetate is carried out for at least about 2 hours, preferably from about 2 hours to about 8 hours, more preferably about 4 hours.

Tegaserod base may be obtained according to any method known in the art, such as the one described in WO 2005/105740, incorporated herein by reference, which comprises a reaction between N-amino-N′-pentylguanidine hydroiodide (AGP-HI) and 5-Methoxy-1H-indole-3-carbaldehyde (5-MICHO) in water under acidic or basic conditions. Alkali metal and alkaline earth metal bases such as sodium hydroxide can be used. In one embodiment, an aqueous solution of AGP-HI is combined with MICHO and sodium hydroxide.

Before extracting tegaserod base with ethyl acetate, the tegaserod base in water may optionally be heated to a temperature of from about room temperature to about reflux temperature. Preferably, the mixture is heated to a temperature of about 40° C. The amount of water used in the reaction mixture is preferably at least about 2 equivalents of the tegaserod base used.

Before combining the mixture of tegaserod base and ethyl acetate with a solution of maleic acid in water, the mixture may optionally be heated to a temperature of about room temperature to about 70° C., more preferably to a temperature of about 60° C. to about 65° C. If the mixture is heated, the process may further comprise cooling the mixture. Preferably, the mixture is cooled to a temperature of about 5° C. to about room temperature, more preferably to a temperature of about 10° C.

Preferably, after the cooling step, the reaction mixture is maintained, while stirring for a period of time of preferably at least about 8 hours, more preferably about 12 hours.

Preferably, the reaction mixture containing Form H2 is maintained for about 10 minutes to about 5 hours, more preferably for about 2 hours.

The sesqui-tegaserod maleate Form H2 can then be recovered by any method known in the art, such as filtering, washing and drying. Form H2 can also be washed with ethyl acetate.

The present invention also provides a method for making tegaserod maleate by preparing sesqui-tegaserod maleate as described above, and converting it to a tegaserod maleate other than the sesqui-tegaserod maleate.

The conversion may be performed according to any method known in the art, such as combining with maleic acid, with or without the presence of an organic solvent.

The obtained tegaserod maleate may be in any crystalline and/or amorphous form.

The present invention further provides a process for preparing the tegaserod maleate crystalline form characterized by an X-ray Diffraction pattern having peaks at 15.7, 16.9, 17.2, 24.1, 24.6 and 25.2±0.2 two theta (designated as Form B), comprising combining sesqui-tegaserod maleate Form H2, n-propanol and maleic acid to obtain a slurry and maintaining the slurry to obtain Form B. In one embodiment, the sesqui-tegaserod maleate Form H2 is slurried in in n-propanol or a mixture thereof with ethyl acetate and then combined with maleic acid to obtain tegaserod maleate Form B. The ratio of tegaserod to maleate in Form B is believed to be 1:1.

Preferably, the reaction mixture obtained after combining the slurry with maleic acid is maintained for preferably at least about 3 hours, more preferably about 5 hours.

Tegaserod maleate Form B may be recovered from the reaction mixture by any method known in the art, such as filtering. The tegaserod maleate may further be washed. Preferably, Form B is washed with n-propanol. The tegaserod maleate may also be dried. The temperature may be increased and/or pressure reduced to accelerate the drying process. The drying process may be carried out at a temperature of about 40° C. to about 60° C., preferably about 50° C. The pressure may be that of a vacuum, i.e., a pressure of less than about 100 mmHg. A vacuum oven can be used.

The invention further provides pharmaceutical formulations comprising sesqui-tegaserod maleate Form H2. The compositions of the invention include powders, granulates, aggregates and other solid compositions comprising sesqui-tegaserod maleate Form H2. In addition, the solid formulations comprising the above form sesqui-tegaserod maleate of the present invention may further include diluents, such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents like calcium carbonate and calcium diphosphate and other diluents known to the pharmaceutical industry. Yet other suitable diluents include waxes, sugars and sugar alcohols like mannitol and sorbitol, acrylate polymers and copolymers, as well as pectin, dextrin and gelatin.

Further excipients that are suitable in the present invention include binders, such as acacia gum, pregelatinized starch, sodium alginate, glucose and other binders used in wet and dry granulation and direct compression tableting processes. Excipients that also may be present in a solid formulation of the above form of sesqui-tegaserod maleate further include disintegrants like sodium starch glycolate, crospovidone, low-substituted hydroxypropyl cellulose and others. In addition, excipients may include tableting lubricants like magnesium and calcium stearate and sodium stearyl fumarate; flavorings; sweeteners; preservatives; pharmacy parenteral (including subcutaneous, intramuscular, and intravenous), inhalant and ophthalmic administration. Although the most suitable route in any given case will depend on the nature and severity of the condition being treated, the most preferred route of the present invention is oral. Dosages may be conveniently presented in unit dosage form and prepared by any of the methods well-known in the art of pharmacy.

Dosage forms include solid dosage forms, like tablets, powders, capsules, suppositories, sachets, troches and losenges as well as liquid suspensions and elixirs. While the description is not intended to be limiting, the invention is also not intended to pertain to true solutions of sesqui-tegaserod maleate whereupon the properties that distinguish the solid form of sesqui-tegaserod maleate are lost. However, the use of the novel forms to prepare such solutions (e.g. so as to deliver, in addition to sesqui-tegaserod maleate, a solvate to said solution in a certain ratio with a solvate) is considered to be within the scope of the invention.

Capsule dosages, of course, will contain the solid composition within a capsule which may be made of gelatin or other conventional encapsulating material. Tablets and powders may be coated. Also, tablets and powders may be coated with an enteric coating. The enteric coated powder forms may have coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

PXRD:

Powder X-ray diffraction (“PXRD”) analysis using a SCINTAG powder X-ray diffiactometer model X'TRA equipped with a solid-state detector. Copper radiation of λ=1.5418 Å was used. The sample was introduced using a round standard aluminum sample holder with round zero background quartz plate in the bottom.

### Example 1

### Preparation of Sesqui-tegaserod Maleate Form H2 Through Tegaserod Base

To a mixture of AGP.HI (112.7 g) in 283 mL of water was added 5-MICHO (45 g) followed by NaOH (52.8 g, 47%) and stirred at room temperature. After three hours, 522 mL of ethyl acetate were added and the mixture stirred for an additional four hours. After phase separation at 40° C. the organic phase was washed with water (3×218 ml), and filtrated under vacuum. The resulting solution was heated to 60° C. and a solution of maleic acid (14.4 g) in 45 mL water was dropped during half hour, and the reaction mixture stirred at the same temperature for an additional two hours. The mixture was cooled to 10° C. during one hour, kept under stirring at the same temperature for 12 hrs and then filtered under vacuum. The wet product was washed twice with 65 ml of ethyl acetate and dried in a vacuum oven at 45° C. for 16 hours to give 85% of the product. Example 2

### Preparation of Sesqui-tegaserod Maleate Form H2

45 gr MICHO were added to a 1 L reactor at RT. A solution of 112.7 gr of AGP HI and 283 ml water was added to the reactor. 52.8 gr of NaOH 47% were added to the mixture while stirring. The mixture was heated to 40° C. and stirred for 12 hrs. 522 ml of Ethyl Acetate were added and the mixture was stirred for 4 hrs.

After phase separation at 40° C. the organic phase was washed with water (3×218 ml), and filtrated under vacuum.

The mixture was heated to 60° C. and a mixture o 14.4 gr of Maleic Acid in 45 ml water was dropped during 5 min.

The mixture was stirred at 60° C. for 2 hrs.

The mixture was cooled to 10° C. during 1 hour, stirred at 10° C. for 13 hrs and then filtered under vacuum. The wet product was washed twice with 65 ml of n-Propanol.

The wet product was dried in a vacuum oven at 45° C.

Yield: 71.2%

### Example 3

### Preparation of Tegaserod Maleate Form B from Sesqui-tegaserod Maleate Form H2

6.9 g of maleic acid were added to a slurry of Sesqui-Tegaserod maleate Form H2 (41.5 g) in 208 ml n-propanol at room temperature. The mixture was stirred for 5 hours at the same temperature, filtered and washed with n-propanol. After drying on vacuum oven at 45° C. for 15 hours the product was analyzed by XRD and found to be Form B (89% yield).

What is claimed is:

1. Sesqui-tegaserod maleate.

2. Sesqui-tegaserod maleate of claim 1, wherein the maleate is isolated.

3. Sesqui-tegaserod maleate of claim 1, wherein the maleate is in crystalline form.

4. The sesqui-tegaserod maleate claim 3, wherein the sesqui-tegaserod maleate is a hydrate.

5. The sesqui-tegaserod maleate of claim 3, wherein the sesqui-tegaserod maleate is a dihydrate.

6. The crystalline form of claim 3, wherein the crystalline form is present in a batch having a polymorphic purity of at least about 90% of said crystalline form.

7. The Sesqui-tegaserod maleate of claim 3 wherein the sesqui-tegaserod maleate is in crystalline form which is characterized by a powder XRD pattern with peaks at about 3.9, 5.1, 5.4, and 14.0±0.2 degrees 2-theta.

8. The Sesqui-tegaserod maleate of claim 7, further characterized by a peak at about 27.0±0.2 degrees 2-theta.

9. The Sesqui-tegaserod maleate of claim 8, further characterized by a powder XRD pattern with peaks at about 10.6, 13.6, 16.5, 22.8 and 29.7±0.2 degrees 2-theta.

10. The Sesqui-tegaserod maleate of claim 9, further characterized by an X ray powder diffraction pattern substantially as depicted in FIG. 1.

11. A process for preparing the crystalline form of claim 1, comprising providing tegaserod base in water; extracting the tegaserod base with ethyl acetate for more than one hour to obtain a mixture; combining the mixture with an aqueous solution of maleic acid.

12. The process of claim 11, wherein the extraction of the base into ethyl acetate is carried out for at least about 2 hours.

13. The process of claim 11, wherein tegaserod base is obtained by a reaction between N-amino-N′-pentylguanidine hydroiodide (AGP-HI) and 5-Methoxy-1 H-indole-3-carbaldehyde (5-MICHO) in water under acidic or basic conditions.

14. The process of claim 13, wherein MICHO is combined with a solution of AGP-HI and water.

15. The process of claim 13, wherein the reaction is carried out under basic condition.

16. The process of claim 15, wherein the base is an alkali metal or alkaline earth metal base.

17. The process of claim 16, wherein the base is NaOH.

18. The process of claim 11, wherein the water containing reaction mixture having tegaserod base is heated before addition of ethyl acetate.

19. The process of claim 18, wherein the mixture is heated to about 40° C.

20. The process of claim 11, wherein the ethyl acetate containing tegaserod maleate is separated from the water at a temperature of about room temperature to about reflux temperature.

21. The process of claim 20, wherein phase separation is carried out at about 40° C.

22. The process of claim 11, wherein the mixture of ethyl acetate and tegaserod base is heated before addition of maleic acid.

23. The process of claim 22, wherein the ethyl acetate is heated to about 60° C.

24. The process of claim 11, wherein the reaction mixture after addition of maleic acid is maintained for about 2 hours.

25. The process of claim 11, wherein the tegaserod maleate is recovered by precipitation.

26. The process of claim 25, wherein precipitation is induced by cooling.

27. The process of claim 26, wherein cooling is carried out to a temperature of about 5° C. to about room temperature.

28. The process of claim 11, wherein the extraction is carried out for about 2 hours to about 8 hours.

29. The process of claim 28, wherein the extraction is carried out for about 4 hours.

30. A pharmaceutical composition comprising crystalline sesqui-tegaserod maleate of claim 30 and a pharmaceutically acceptable excipient.

31. A method for preparing a pharmaceutical composition comprising combining the crystalline sesqui-tegaserod maleate of claim 1 with a pharmaceutically acceptable excipient.

32. A method of treating irritable bowl syndrome comprising administering the pharmaceutical composition of claim 31 to a mammal.

33. A process for preparing tegaserod maleate comprising converting the sesqui-tegaserod maleate of claim 3 to tegaserod maleate.

34. The process of claim 33, wherein the tegaserod maleate obtained has a ratio of 1:1 tegaserod to maleic acid.

35. A process for preparing crystalline tegaserod maleate characterized by an X-ray Diffraction pattern having peaks at 15.7, 16.9, 17.2, 24.1, 24.6 and 25.2±0.2 two theta, comprising combining sesqui-tegaserod maleate with n-propanol and maleic acid, and maintaining the slurry to obtain the crystalline form.

36. The process of claim 35, wherein the slurry is maintained for about 3 to about 5 hours.

37. The process of claim 36, wherein the slurry is maintained for about 5 hours.

38. The process of claim 35, further comprising recovering the tegaserod maleate.

39. The process of claim 11, wherein the Sesqui-tegaserod maleate is in crystalline form which is characterized by a powder XRD pattern with peaks at about 3.9, 5.1, 5.4, and 14.0±0.2 degrees 2-theta.

40. The process of claim 39, wherein the Sesqui-tegaserod maleate is further characterized by a peak at about 27.0±0.2 degrees 2-theta.

41. The process of claim 40, wherein the Sesqui-tegaserod maleate is further characterized by a powder XRD pattern with peaks at about 10.6, 13.6, 16.5, 22.8 and 29.7±0.2 degrees 2-theta.
